(19) United States
(12) Patent Application Publication
(10) Pub. No.: US 2010/0143501 A1
(43) Pub. Date: Jun. 10, 2010

(54) SOLID COMPOSITION COMPRISING A CLAY SUCH AS KAOLIN

(75) Inventors: Stefanie Christian, Herrenberg (DE); Michael Harkenthal, Buehl (Baden) (DE); Ulrich Thomas Schobert, Herrenberg (DE)

Correspondence Address: GlaxoSmithKline;GLOBAL PATENTS -US, UW2220, P. O. BOX 1539, KING OF PRUSSIA, PA, 19406-0939, US

(73) Assignee: GLAXOSMITHKLINE CONSUMER HEALTHCARE GMBH & CO KG, Buehl (DE)

(21) Appl. No.: 12/598,502

(22) PCT Filed: May 7, 2008
(86) PCT No.: PCT/EP2008/055589

§ 371 Date: Nov. 2, 2009

(30) Foreign Application Priority Data
May 9, 2007 (GB) 0708929.5

(51) Int. Cl. A61K 33/06; A61K 8/26; A61Q 19/00; A61P 1/00

(52) U.S. Cl. 424/684

(57) ABSTRACT

The present invention relates to a therapeutic composition comprising at least one clay, at least one disintegrant, at least one lubricant and at least one binding agent, particularly wherein the at least one clay is kaolin, and in particular to a palatable clay composition in the form of a tablet, and to a process for the preparation of the therapeutic composition

The present invention relates to a therapeutic composition comprising at least one clay, at least one disintegrant, at least one lubricant and at least one binding agent, particularly wherein the at least one clay is kaolin, and in particular to a palatable clay composition in the form of a tablet, and to a process for the preparation of the therapeutic composition.

Clay compositions, for example comprising a natural clay such as healing earth or kaolin have been known since ancient times. Healing earth, comprising a range of minerals and trace elements, has been used for treating many ailments and disorders of the body. Healing earth is available commercially as a powder for external application, such as the powder preparation, Heilerde, sold under the brand name Bullrich. It is also sold in capsule form for internal application, under the Bullrich brand.

Kaolin, another natural clay, has been used as an adsorbent for external cleansing of the skin and is also known for internal cleansing of the body. Kaolin is also known as Bolus alba, china clay, porcelain clay, white bole, and argilla and has the approximate chemical formula of H₂Al₂Si₂O₈ (H₂O). Finely divided particles of kaolin yield a very large surface area and are known to adsorb a wide variety of materials. For internal cleansing of the body, to be effective therapeutically, kaolin is normally administered at a dose of from about 2 to about 20 g per day. For external cleansing of the body, to be effective therapeutically, a dose of from 2 to about 80 g per day may be administered. For example, a dose of from about 2 to about 20 g may be administered, in an amount from one to four applications daily, depending on the nature and amount of external cleansing required, for example the area of skin to be cleansed. When taken in capsule form, a daily dose may require a large number of capsules to be consumed, for example a daily dose of the capsules sold under the Bullrich brand consists of six capsules.

WO 00/075079 relates to an effervescent therapeutic composition containing a clay useful for internal or external use. The composition comprises between 40 to 85% of single clay minerals, together with effervescent ingredients.

DE 20009867 U1 relates to the composition as described in WO 00/075079.

Whilst therapeutic compositions comprising clays are known in the art, there nevertheless remains a need for improved therapeutic clay compositions. It is the aim of the present invention to provide such an improved composition. A composition according to the present invention will comprise one or more of the following advantages:

(a) is palatable and has improved organoleptic properties e.g. improved smooth taste and mouthfeel;
(b) is less messy and easier to handle than commercially available powder preparations;
(c) does not have the problem of foaming associated with the use of effervescent agents;
(d) when in the form of a tablet, provides the required dose in one tablet so that only one daily dose is required; and
(e) has an improved (shorter) disintegration time.

According to the present invention there is provided a therapeutic composition comprising at least one clay, at least one disintegrant, at least one lubricant and at least one binding agent.

In one aspect, a therapeutic composition according to the present invention is a composition for oral administration, which may be formulated so as to be palatable. In one aspect, the composition of the present invention is for internal cleansing of the body.

In another aspect, a therapeutic composition according to the present invention is for external application to the body. In one aspect, the composition of the present invention is for external cleansing of the body.

In another aspect, a therapeutic composition according to the present invention is for internal or external cleansing of the body.

In one aspect, a composition according to the present invention is in the form of a tablet. In a further aspect, a tablet for oral administration is a swallow tablet or a tablet to be chewed. In another aspect, the composition is a non-effervescent composition, for example a non-effervescent tablet.

A composition of the present invention may be dispersed in a liquid prior to ingestion or prior to external administration. For example, the composition may be dispersed in a suitable amount of water for oral consumption. In an alternative embodiment, the composition may be mixed with water to form a paste for external application.

A composition according to the present invention comprises at least one clay. In one aspect, the clay component for use in the composition comprises kaolin of a pharmaceutically acceptable grade, as may be obtained, for example, from Merck Pharmaceuticals or from Amberger Kaolin Werke. Kaolin for use in a composition of the present invention may be heavy or light kaolin.

In one aspect, the at least one clay, such as kaolin, for use in the invention, is present in the composition in an amount of from about 30.0 to about 70.0% w/w, for example from about 40.0 to about 60.0% w/w.

A composition according to the invention comprises at least one disintegrant. Suitable disintegrants for use in the invention include commercially available disintegrants commonly used in oral dosage forms. In one aspect, the at least one disintegrant is selected from: sodium starch glycolate, crosslinked povidone, calcium carboxymethylcellulose and crosslinked carboxymethylcellulose, or is a combination of two or more thereof.

In one aspect, the at least one disintegrant is present in the composition in an amount of from about 1.0 to about 8.0% w/w, for example from about 2.0 to about 5.0% w/w.

A composition according to the invention comprises at least one lubricant. Suitable lubricants for use in the invention include commercially available lubricants commonly used in oral dosage forms. In one aspect, the at least one lubricant is selected from: fumaric acid, magnesium lauryl sulphate, solid polyethylene glycol (PEG), sodium lauryl sulphate, calcium behenate, calcium arachinate and sodium stearyl sulphate, or is a combination of two or more thereof.

In one aspect, the at least one lubricant is present in the composition in an amount of from about 1.0 to about 8.0% w/w, for example from about 2.0 to about 5.0% w/w.

A composition according to the invention comprises at least one binding agent. Suitable binding agents for use in the invention include commercially available binding agents commonly used in oral dosage forms. In one aspect, the at least one binding agent is selected from: microcrystalline cellulose, sorbitol, mannitol, polyvinylpyrrolidone (PVP), calcium phosphate, saccharose (sucrose) and lactose, or is a combination of two or more thereof.

In one aspect, the at least one binding agent is present in a composition in an amount of from about 1.0 to about 40.0% w/w, for example from about 5.0 to about 40.0% w/w, or from about 5.0 to about 30.0% w/w.

A composition of the invention optionally further comprises a colouring agent which may be commercially available. In one aspect the optional colouring agent is selected from: red iron oxide, yellow iron oxide and a combination thereof.

In one aspect, the colouring agent may be present in a composition in an amount of from about 1.0 to about 5.0% w/w, for example from about 2.0 to about 4.0% w/w.

A composition of the invention optionally further comprises ingredients typically used in oral dosage forms such as a flavouring agent, compression aid; preservative; wetting agent; bulking agent; adhesive or a sweetening agent.

The invention also provides a process for the preparation of a composition of the invention, which process comprises admixing at least one clay, at least one disintegrant, at least one lubricant, at least one binding agent, and optionally any other desired ingredient, to form a mixture or blend, using techniques commonly used in the field, and optionally tableting the mixture or blend.

When a composition is in the form of a tablet, the tablet is compressed to provide a hardness of greater than about 100N, for example from about 100 to about 140N. It has been found that a tablet hardness in the range of from about 100 to about 140N facilitates rapid disintegration and ease of packaging and handling of the tablet.

Tableting may be achieved by techniques commonly used in the field of tablet making In one aspect, a composition according to the invention as herein described undergoes rapid disintegration in the presence of water. A composition, for example when in the form of a tablet, disintegrates in water within about 15 minutes or less prior to administration.

In one aspect, a composition of the present invention has a disintegration time in water of about 5 to about 15 minutes, for example of about 8 to about 12 minutes.

In one aspect, a tablet composition according to the present invention comprises a therapeutic dose of from about 2 to about 5 g of clay. In one aspect, a tablet comprising such a dose will comprise one or more score lines, i.e. break lines or grooves, to enable ease of breaking of the tablet, as may be necessary for ease of dosing, in particular if the tablet is of a large size. In a further aspect, the tablet comprises a plurality of score lines.

Score lines are typically introduced during compression of the powder mixture by means of a suitably shaped punch (mould).

### EXAMPLES

The following Examples are illustrative of the invention.

### Example 1

The following tablet formulation according to the present invention was prepared.

| | | Amount | Percentage |
|---|---|---|---|
| Ingredient | Function | mg/tablet | w/w |
| Kaolin (heavy) | Active ingredient | 3000 | 50% |
| Microcrystalline cellulose | Binder | 1200 | 20% |
| Sorbitol | Binder/Flavor | 780 | 13% |
| Mannitol | Binder/Flavor | 420 | 7% |
| Calcium arachinate/behenate | Lubricant | 300 | 5% |
| Sodium starch glycolate | Disintegrant | 180 | 3% |
| Iron oxide, red | Colour | 120 | 2% |

Kaolin, microcrystalline cellulose, mannitol, sorbitol and iron oxide were roughly weighed and sieved. These components (kaolin, microcrystalline cellulose, mannitol, sorbitol and iron oxide) were weighed accurately into a container and blended together in a container mixer. ⅓ of the total amount of calcium arachinate/behenate was sieved and added to the mixture and a second blending step was carried out. Subsequently, the powder mixture was compressed in a dry granulation step using a roller-compactor. The resulting ribbons were granulated with a granulation unit connected to the roller compactor. The remaining ⅔ of the total amount of calcium arachinate/behenate, and sodium starch glycolate were roughly weighed, sieved, added to the granulate and mixed in a container mixer.

The powder mixture was compressed to tablets on a rotary tablet press.

### Examples 2-5

The following tablet formulations according to the present invention can be prepared as follows.

The active ingredient, the binders and the colouring agent are roughly weighed and sieved. These components (active ingredient, binders and colouring) are weighed accurately into a container and blended in a container mixer. ⅓ of the total amount of lubricant is sieved and added to the mixture and a second blending step is carried out. Subsequently, the powder mixture is compressed in a dry granulation step using a roller-compactor. The resulting ribbons are granulated with a granulation unit connected to the roller compactor. The remaining ⅔ of the total amount of lubricant and the disintegrant are roughly weighed, sieved, added to the granulate and mixed in a container mixer.

The powder mixture is compressed to tablets on a rotary tablet press.

### Example 2

| | | Amount | Percentage |
|---|---|---|---|
| Ingredient | Function | mg/tablet | w/w |
| Kaolin (heavy) | Active Ingredient | 2700 | 45% |
| Microcrystalline cellulose | Binder | 1200 | 20% |
| Povidone | Binder | 300 | 5% |
| Sorbitol | Binding agent/ | 600 | 10% |
| | Flavour | | |
| Mannitol | Binding agent/ | 600 | 10% |
| | Flavour | | |
| Calcium arachinate/behenate | Lubricant | 300 | 5% |
| Povidone crosslinked | Disintegrant | 180 | 3% |
| Iron oxide | Colour | 120 | 2% |

### Example 3

| | | Amount | |
|---|---|---|---|
| Ingredient | Function | mg/tablet | Percentage |
| Kaolin (heavy) | Active ingredient | 3000 | 50% |
| Microcrystalline cellulose | Binder | 1200 | 20% |
| Sucrose | Binder/Flavor | 1200 | 20% |
| Calcium arachinate/behenate | Lubricant | 300 | 5% |
| Sodium starch glycolate | Disintegrant | 180 | 3% |
| Iron oxide, red | Colour | 120 | 2% |

### Example 4

| | | Amount | |
|---|---|---|---|
| Ingredient | Function | mg/tablet | Percentage |
| Kaolin (heavy) | Active ingredient | 3000 | 50% |
| Microcrystalline cellulose | Binder | 1800 | 30% |
| Sucrose | Binder/Flavor | 600 | 10% |
| Calcium arachinate/behenate | Lubricant | 300 | 5% |
| Sodium starch glycolate | Disintegrant | 180 | 3% |
| Iron oxide, red | Colour | 120 | 2% |

### Example 5

| | | Amount | |
|---|---|---|---|
| Ingredient | Function | mg/tablet | Percentage |
| Kaolin (heavy) | Active ingredient | 3000 | 50% |
| Microcrystalline cellulose | Binder | 1200 | 20% |
| Sucrose | Binder/Flavor | 600 | 10% |
| Lactose | Binder | 600 | 10% |
| Calcium arachinate/behenate | Lubricant | 300 | 5% |
| Sodium starch glycolate | Disintegrant | 180 | 3% |
| Iron oxide, red | Colour | 120 | 2% |

### Example 6

### Measurement of Disintegration Time

Tablet disintegration may be measured with the disintegration apparatus and testing method described in Ph. Eur. 01/2006; 2.9.1. “Disintegration of tablets and capsules”. The following is an Example of the process used to measure disintegration time of the compositions of the present invention:

Three tablets were employed and placed one each into the three cylindrical transparent tubes of the bigger basket-rack assembly of the disintegration tester (type: Erweka ZT32). The tablets were covered with the respective plastic discs. The water bath of the apparatus was heated to a temperature of 36 C. From time to time the apparatus was stopped to monitor the disintegration process, because the disintegration medium (water) became cloudy while the tablets disintegrated. The disks covering the tablets were lifted by means of tweezers to enable a visual control of the disintegration process and the status of disintegration respectively. The time needed to disintegrate was determined using a clock.

The disintegration time is defined as: the time elapsed when all three tablets are completely disintegrated and no residue is observed in the tubes of the basket.

The disintegration time of Example 1 was measured according to the above procedure. Example 1 was found to have a disintegration time of 10 minutes.

### Example 7

### Measurement of Tablet Hardness

Tablet hardness may be defined as the resistance to crushing of tablets, measured by the force needed to disrupt them by crushing

Tablet hardness may be measured with the disintegration apparatus and testing method described in Ph. Eur. 01/2008; 2.9.8. “Resistance to crushing of tablets”. The following is an Example of the process used to measure tablet hardness of the compositions of the present invention:

Five tablets were tested using the high speed hardness tester HDT 1 V.3 (made by, and purchased from CGS GmbH). The tablets were placed between the jaws and subsequently the “Test for crushing strength” was chosen from the menu of the DOS-based software QPBT1.EXE (also made by, and purchased from CGS GmbH).

The tablet hardness in Newtons (N) is calculated as the mean value obtained for the five tablets, and is expressed as a range, (taking into account a relative standard deviation of 5-10%).

The tablet hardness of Example 1 was measured according to the above procedure. Example 1 was found to have a tablet hardness of 100-130 N.

1. A therapeutic composition comprising at least one clay, at least one disintegrant, at least one lubricant and at least one binding agent.

2. A therapeutic composition according to claim 1 which is in the form of a tablet for oral delivery

3. A therapeutic composition according to claim 1, wherein the at least one clay comprises kaolin.

4. A therapeutic composition according to claim 1, wherein the at least one clay is kaolin as the sole clay component.

5. A therapeutic composition according to claim 1, wherein the at least one clay is present in an amount of from about 30.0 to about 70.0% w/w.

6. A therapeutic composition according to claim 1, wherein the at least one disintegrant is selected from: sodium starch glycolate, crosslinked povidone, calcium carboxymethylcellulose and crosslinked carboxymethylcellulose, or is a combination of two or more thereof.

7. A therapeutic composition according to claim 6, wherein the at least one disintegrant is present in an amount of from about 1.0 to about 8.0% w/w.

8. A therapeutic composition according to claim 1, wherein the at least one lubricant is selected from: fumaric acid, magnesium lauryl sulphate, solid polyethylene glycol, sodium lauryl sulphate, calcium behenate, calcium arachinate and sodium stearyl sulphate, or is a combination of two or more thereof.

9. A therapeutic composition according to claim 8, wherein the at least one lubricant is present in an amount of from about 1.0 to about 8.0% w/w.

10. A therapeutic composition according to claim 1, wherein the at least one binding agent is selected from: microcrystalline cellulose, sorbitol, mannitol, polyvinylpyrrolidone (PVP), calcium phosphate, saccharose (sucrose) and lactose, or is a combination of two or more thereof.

11. A therapeutic composition according to claim 10, wherein the at least one binding agent is present in an amount of from about 1.0 to about 40.0% w/w.

12. A therapeutic composition according to claim 1, further comprising at least one colouring agent in an amount from about 1.0 to about 5.0% w/w.

13. A therapeutic composition according to claim 12 wherein the at least one colouring agent is selected from: red iron oxide, yellow iron oxide and a combination thereof.

14. A therapeutic composition according to claim 1, wherein the tablet comprises one or more score lines.

15. A therapeutic composition according to claim 1, wherein the tablet comprises a hardness of greater than about 100N.

16. (canceled)

17. A method of internally or externally cleansing the body in a human in need thereof, comprising administering orally or topically to said human and effective amount of the therapeutic composition according to claim 1.

18. A therapeutic composition according to claim 2, wherein the disintegration time of the composition in water is about 15 minutes or less.

19. A process for the preparation of a therapeutic composition according to claim 1, which process comprises admixing at least one clay, at least one disintegrant, at least one lubricant and at least one binding agent to form a mixture or blend and optionally tableting the mixture or blend.

20. A therapeutic composition for oral delivery in the form of a tablet comprising:
1) at least one clay;
2) at least one disintegrant selected from the group consisting of sodium starch glycolate, crosslinked povidone, calcium carboxymethylcellulose and crosslinked carboxymethylcellulose, and combinations of two or more thereof;
3) at least one lubricant selected from the group consisting of fumaric acid, magnesium lauryl sulphate, solid polyethylene glycol, sodium lauryl sulphate, calcium behenate, calcium arachinate, sodium stearyl sulphate, and combinations of two or more thereof;
4) and at least one binding agent least one binding agent selected from the group consisting of microcrystalline cellulose, sorbitol, mannitol, polyvinylpyrrolidone (PVP), calcium phosphate, saccharose (sucrose) and lactose, and combinations of two or more thereof; and
5) optionally a colouring agent, flavouring agent, compression aid, preservative, wetting

21. A therapeutic composition according to claim 20 wherein the clay is kaolin, present in an amount of about 30 to 70% w/w.

22. A therapeutic composition according to claim 21 wherein the binder is microcrystalline cellulose, and the disintegrant is sodium starch glycolate or crosslinked povidone.
